(19) United States
(12) Patent Application Publication
(10) Pub. No.: US 2005/0208096 A1
(43) Pub. Date: Sep. 22, 2005

(54) Color-fast radiation sterilized implants and precursors thereof

(76) Inventor: Shalaby W. Shalaby, Anderson, SC (US)

Correspondence Address: LEIGH P. GREGORY;ATTORNEY AT LAW, PO BOX 168, CLEMSON, SC, 29633-0168, US

(21) Appl. No.: 11/083,133

(22) Filed: Mar. 17, 2005

Related U.S. Application Data
(60) Provisional application No. 60/553,917, filed on Mar. 17, 2004.

(51) Int. Cl. A61K 9/14; A61F 2/06

(52) U.S. Cl. 424/423; 623/1.11

(57) ABSTRACT

This invention is directed to color-fast radiation sterilized surgical implants formed of absorbable/biodegradable or non-absorbable polymers and a lipophilic disperse-type dye.

The present application claims the benefit of prior provisional application, U.S. Ser. No. 60/553,917, filed Mar. 17, 2004.

### FIELD OF THE INVENTION

This invention is directed to dyed, absorbable, surgical implants, which can be radiation or radiochemically sterilized without compromising the color intensity or shade.

### BACKGROUND OF THE INVENTION

Evolution of synthetic absorbable/biodegradable polymers has been reviewed in a recent article [Shalaby, S. W. and Burg, K. J. L, Chapter 1 in Absorbable & Biodegradable Polymers, (Shalaby & Burg, Eds.) CRC Press, New York, 2004]. It has been noted that interest in synthetic absorbable polymers has grown considerably over the past three decades, principally because of their transient nature when used as biomedical implants or drug carriers. The genesis of absorbable polymers was driven by the need to replace the highly tissue reactive, absorbable, collagen-based sutures with synthetic polymers, which elicit milder tissue response. This led to the early development of polyglycolide as an absorbable polyester suture. In spite of the many polymeric systems investigated as candidates for absorbable implants and drug carriers, ester-based polymers maintain an almost absolute dominance among clinically used systems and others that are under investigation.

In addition to the ester-based polymers, a great deal of research activity has been directed to other types of absorbable polymers, but the clinical relevance of their properties practically halted their evolution beyond the exploratory phase. Typical examples of these include polyanhydrides, polyphosphazenes, and certain polyamidoesters.

In spite of their fast-growing clinical significance, the vast majority of the absorbable polymer devices are sterilized by ethylene oxide due to their exceptional propensity to degrade during a typical high-energy radiation sterilization cycle requiring a nominal dose of 25 kGy. Only a small fraction of commercially available sutures that's intended uses call for radiolytic degradation are sterilized by high-energy radiation such as gamma rays and electron beam. Most pertinent to the present invention is the fact that the radiation sterilized devices are only available as dye-free products since the only two available dyes, namely, D&C Violet #2 (Color Index # 60725) and D&C Green #6 (Color Index No. 61565), used to date, undergo substantial discoloration, change in color or total bleaching. This can be attributed to chemical interaction with the reactive polymer matrix in the presence of high energy radiation and traces of oxygen present in the polymer and/or in the sealed package.

To minimize or eliminate reliance on ethylene oxide and exploit reliability and assured sterility using radiation for absorbable polymers and particularly those used in tissue engineering, Shalaby and coworkers developed the radiochemical sterilization (RC-S) process [U.S. Pat. No. 5,5422,065 (1995); Trans. Soc. Biomater., 24, 157 (2001)]. The RC-S process represents a novel approach to the sterilization of certain mechanical devices, such as those made of absorbable polyesters, that are sensitive to high-energy radiation delivered at the traditional dose of 25 kGy. RC-S is a hybrid process encompassing the attributes of chemical and high-energy radiation sterilization without the drawbacks associated with the use of the parent processes. RC-S entails the use of about 5 to 7.5 kGy of gamma radiation and a poly-formaldehyde package insert capable of a radiolytically controlled release of formaldehyde in a hermetically sealed package under dry nitrogen. The process has been applied successfully to absorbable sutures without compromising their clinically relevant properties, such as their in vivo breaking strength retention (BSR). Typical BSR data of radiochemically sterilized suture braids and controls have shown that using the RC-S protocol does not compromise the sutures clinically critical properties [Trans. Soc. Biomater., 24, 157 (2001)].

Recent developments in high-energy radiation sterilization, as in the above noted radiochemical sterilization (RC-S), and consistent demands by surgeons for dyed or colored absorbable medical devices, which can be easily recognized in bloody surgical fields, evoked the need for an acceptable dye that maintains its original color in the absorbable polymeric device during high-energy radiation sterilization, where the two commonly used D&C Violet #2 and D&C Green #6 failed to do so. In addition, dye instability in currently available, dyed absorbable devices can be encountered during cold plasma sterilization leading to unacceptable color changes or discoloration. This provided a strong incentive to explore the availability of special dyes for use in radiation- or plasma-sterilized absorbable devices. Accordingly, this invention deals with color-fast (capable of maintaining its color under prevailing conditions) radiation or plasma sterilized solid absorbable implants wherein the color-fastness is achieved by incorporating lipophilic dispersed dye or microparticulate/nano-particulate inorganic pigments.

### SUMMARY OF THE INVENTION

This invention deals, in general, with dyed or pigmented absorbable/biodegradable polymeric surgical implants which can be sterilized by high-energy radiation or cold gas plasma without compromising the intensity or shade of dye or pigments therein. Such surgical implant formulations are referred to herein as color-fast implants. One aspect of this invention deals with color-fast radiation-sterilized surgical implants comprising an absorbable/biodegradable polymer and lipophilic, disperse-type organic drugs such as D&C Yellow 11 (Color Index Number 4700) or a micro- or nano-particulate inorganic pigment, such as an iron oxide wherein the sterilizing radiations comprise gamma rays, an electron beam or part of a cold gas plasma. Another aspect of this invention deals with color-fast radiation sterilized absorbable surgical implants wherein the sterilization is achieved radiochemically by a combination of gamma rays or electron beam and radiolytically generated formaldehyde from an unstabilized polyformaldehyde. Another aspect of this invention deals with color-fast radiation sterilized surgical implants comprising an absorbable/biodegradable polymer comprising a disperse dye such as D&C Yellow 11 and a polyester made from at least one cyclic monomer selected from the group represented by l -lactide, glycolide, p-dioxanone, 1,5-dioxepan-2-one, a morpholine-2,5-dione and ε-caprolactone wherein the polyester is made using a trifunctional initiator selected from the group represented by trimethylolpropane, triethanolamine, and pentaerythritol. Another aspect of this invention deals with color-fast radiation-sterilized absorbable/biodegradable surgical implants comprising a disperse dye such as D&C Yellow 11 and an absorbable segmented/block copolymer wherein the segmented/block copolymer is made by grafting at least one cyclic polymer selected from the group represented by l-lactide, glycolide, p-dioxanone, 1,5-dioxepan-2-one, a morpholine-2,5-dione and ε-caprolactone onto one of the polymeric initiators selected from the group represented by poly(alkylene succinate) glycol, polyalkylene glycol, copolymeric polyalkylene glycol, and polytrimethylene carbonate glycol. In a typical example, the polymeric initiator is made by ring-opening polymerization of at least one monomer selected from the group represented by trimethylene carbonate, ε-caprolactone, and glycolide in the presence of trimethylolpropane. Another aspect of this invention addresses a color-fast radiation sterilized surgical implant wherein the implant is an absorbable thread in a medical device or component thereof such as a microfilament component of a medical device, an electrospun micro-/nano-fiber-based construct, a coated, twisted multifilament yarn, coated braided multifilament sutures, monofilament sutures, a molded internal bone-fixation device, a knitted mesh, a woven mesh, a knitted tape, or a woven tape.

Another aspect of this invention deals with color-fast radiochemically (using a combination of a low dose of a high-energy radiation, such as an electron beam or gamma rays and radiolytically generated formaldehyde) or radiation (using about 25-40 kGy of high-energy radiation) sterilized medical device, such as a surgical suture or mesh comprising a disperse-type dye, such as D&C Yellow No. 11, and a non-absorbable polymer selected from the group represented by polypropylene, polyethylene terephthalate, segmented copolymers of polytetramethylene terephthalate and polytetramethylene oxide, segmented polycarbonate urethanes, and segmented polyether urethanes.

### DETAILED DESCRIPTION OF THE INVENTION

When medical devices, and particularly surgical sutures and meshes colored with a disperse-type dye, are sterilized by high-energy radiation, such as gamma rays and E-beam, at a nominal dose of about 25 kGy, the color of the sterilized device is substantially compromised as it undergoes a total radiation-bleaching or change in color intensity and hue. Such changes may be associated with structural changes in the dye or interaction with the polymeric matrix, which in turn, may present a safety issue. The increased reliance of radiation and forthcoming radiochemical sterilization of colored medical devices, and particularly absorbable ones, makes it quite desirable to have available radiation-resistant colorant and hence, color-fast medical devices subject of this invention. A preferred aspect of this invention deals with D&C Yellow No. 11 as the disperse-type dye present in medical devices, such as absorbable sutures and meshes made of polyesters and copolyesters, non-absorbable sutures, such as polypropylene and other related surgical devices made of absorbable and non-absorbable polymers.

This invention deals, in general, with dyed or pigmented absorbable/biodegradable polymeric surgical implants which can be sterilized by high-energy radiation or cold gas plasma without compromising the intensity or shade of dye or pigments therein. Such surgical implant formulations are referred to herein as color-fast implants. One aspect of this invention deals with color-fast radiation-sterilized surgical implants comprising an absorbable/biodegradable polymer and lipophilic, disperse-type organic drugs such as D&C Yellow 11 (Color Index Number 4700) or a micro- or nano-particulate inorganic pigment, such as an iron oxide wherein the sterilizing radiations comprise gamma rays, an electron beam or part of a cold gas plasma. Another aspect of this invention deals with color-fast radiation sterilized absorbable surgical implants wherein the sterilization is achieved radiochemically by a combination of gamma rays or electron beam and radiolytically generated formaldehyde from an unstabilized polyformaldehyde. Another aspect of this invention deals with color-fast radiation sterilized surgical implants comprising an absorbable/biodegradable polymer comprising a disperse dye such as D&C Yellow 111 and a polyester made from at least one monomer selected from the group represented by l-lactide, glycolide, p-dioxanone, 1,5-dioxepan-2-one, a morpholine-2,5-dione and ε-caprolactone wherein the polyester is made using a trifunctional initiator selected from the group represented by trimethylolpropane, triethanolamine, and pentaerythritol. Another aspect of this invention deals with color-fast radiation-sterilized absorbable/biodegradable surgical implants comprising a disperse dye such as D&C Yellow 11 and an absorbable segmented/block copolymer wherein the segmented/block copolymer is made by grafting at least one cyclic polymer selected from the group represented by l-lactide, glycolide, p-dioxanone, 1,5-dioxepan-2-one, a morpholine-2,5-dione and ε-caprolactone onto one of the polymeric initiators selected from the group represented by poly(alkylene succinate) glycol, polyalkylene glycol, copolymeric polyalkylene glycol, and polytrimethylene carbonate glycol. In a typical example, the polymeric initiator is made by ring-opening polymerization of at least one monomer selected from the group represented by trimethylene carbonate, ε-caprolactone, and glycolide in the presence of trimethylolpropane. Another aspect of this invention addresses a color-fast radiation sterilized surgical implant wherein the said implant is an absorbable thread such as a microfilament component of a medical device, an electrospun micro-/nano-fiber-based construct, a coated, twisted multifilament yarn, coated braided multifilament sutures, monofilament sutures, a molded internal bone-fixation device, a knitted mesh, a woven mesh, a knitted tape, or a woven tape.

Another aspect of this invention deals with color-fast radiochemically (using a combination of a low dose of a high-energy radiation, such as an electron beam or gamma rays and radiolytically generated formaldehyde) or radiation (using about 25-40 kGy of high-energy radiation) sterilized medical device, such as a surgical suture or mesh comprising a disperse-type dye, such as D&C Yellow No. 11, and a non-absorbable polymer selected from the group represented by polypropylene, polyethylene terephthalate, segmented copolymers of polytetramethylene terephthalate and polytetramethylene oxide, segmented polycarbonate urethanes, and segmented polyether urethanes.

Additional illustrations of the present invention are provided by the following specific examples.

### EXAMPLE 1

### Preparation Radiochemical Sterilization, and Color Evaluation of Monofilament Sutures of Segmented 75/25 Glycolide/ε-Caprolactone Copolymer

A segmented copolyester made of 75/25 glycolide/ε-caprolactone was prepared, isolated, purified, and characterized as described in U.S. Pat. No. 6,498,229 (2002). The ground polymer was mixed with 0.1 weight percent of D&C Violet 11, spun, and oriented into a monofilament suture as described in U.S. Pat. No. 6,498,229 (2002). The suture was transferred to a foil pack and hermetically sealed under dry nitrogen. The package contained a small amount of unstabilized Celcon M-90® as a source of radiolytically generated formaldehyde. The suture package was irradiated with 5 kGy of gamma rays. Shortly after two weeks following sterilization, the suture was isolated and evaluated visually and spectro-scopically for color retention. Color evaluation revealed no discernable changes in shade and intensity.

### EXAMPLE 2

### Preparation Radiochemical Sterilization, and Color Evaluation of Dyed, Braided Multifilament Suture of 95/5 Poly(glycolide-co-l-lactide)

A copolymer of 95/5 glycolide/l-lactide was prepared, isolated, purified, and characterized as described for this class of polymers in U.S. Pat. No. 4,741,337 (1988). The ground polymer was mixed with 0.1 weight percent of D&C Violet 11 and spun-drawn into multifilament yearn using a ¾″ single screw extruder equipped with a 25-hole die. The drawn multifilament yarn was converted to size 2-0 braided suture. The braids were radiochemically sterilized and evaluated visually and spectroscopically for color retention as described in Example No. 8. Color evaluation revealed no discernable change in shade and intensity.

### EXAMPLE 3

### Preparation and Radio-sterilization and Color Evaluation of Dyed, Braided Multifilament Suture of Segmented 88/12 l-Lactide/Trimethylene Copolymer

A segmented copolymer of 88/12 l-lactide/trimethylene carbonate was prepared as described in U.S. Pat. No. 6,342,065 (2002). The purified polymer pellets were mixed with D&C Yellow 11 at 0.1 weight percent and extruded into multifilament yarn as described in Example 2. Size 2-0 braided sutures were constructed from the multifilament yarn. The braids were sterilized under nitrogen in a dry, hermetically sealed package using 25 kGy of gamma radiation. The sterilized braids were evaluated visually and spectroscopically for color retention shortly after two weeks following irradiation. Color evaluation revealed no discernable change in shade and intensity.

Preferred embodiments of the invention have been described using specific terms and devices. The words and terms used are for illustrative purposes only. The words and terms are words and terms of description, rather than of limitation. It is to be understood that changes and variations may be made by those of ordinary skill art without departing from the spirit or scope of the invention, which is set forth in the following claims. In addition it should be understood that aspects of the various embodiments may be interchanged in whole or in part. Therefore, the spirit and scope of the appended claims should not be limited to descriptions and examples herein.

1. A color-fast, sterilized surgical implant made by the process comprising the steps of:
forming a colored surgical implant from a polymer and a lipophilic, dispersed organic dye; and
sterilizing the surgical implant employing a radiation source.

2. The color-fast, sterilized surgical implant set forth in claim 1 wherein the polymer comprises an absorbable/biodegradable polymer.

3. The color-fast, sterilized surgical implant set forth in claim 2 wherein the absorbable/biodegradable polymer comprises a polyester made from at least one cyclic monomer selected from the group represented by l-lactide, glycolide, p-dioxanone, 1,5-dioxepan-2-one, a morpholine-2,5-dione and ε-caprolactone.

4. The color-fast, sterilized surgical implant set forth in claim 3 wherein the polyester is made using a trifunctional initiator selected from the group consisting of trimethylolpropane, triethanolamine, and pentaerythritol.

5. The color-fast, sterilized surgical implant set forth in claim 2 wherein the absorbable/biodegradable polymer comprises a segmented/block copolymer.

6. The color-fast, sterilized surgical implant set forth in claim 5 wherein the segmented/block copolymer is made by grafting at least one cyclic polymer selected from the group consisting of l-lactide, glycolide, p-dioxanone, 1,5-dioxepan-2-one, a morpholine-2,5-dione and ε-caprolactone onto a polymeric initiator selected from the group consisting of poly(alkylene succinate) glycol, polyalkylene glycol, copolymeric polyalkylene glycol, and polytrimethylene carbonate glycol.

7. The color-fast, sterilized surgical implant set forth in claim 6 wherein the polymeric initiator is made by ring-opening polymerization of at least one monomer selected from the group consisting of trimethylene carbonate, ε-caprolactone, and glycolide in the presence of a trifunctional initiator selected from the group consisting of trimethylolpropane, triethanolamine, and pentaerythritol.

8. The color-fast, sterilized surgical implant set forth in claim 1 wherein the polymer comprises a non-absorbable polymer.

9. The color-fast, sterilized surgical implant set forth in claim 8 wherein the non-absorbable polymer is selected from the group consisting of polypropylene, polyethylene terephthalate, segmented copolymers of polytetramethylene terephthalate and polytetramethylene oxide, segmented polycarbonate urethanes, and segmented polyether urethanes.

10. The color-fast, sterilized surgical implant set forth in claim 2 in the form of an absorbable thread comprising a medical device or component thereof selected from the group consisting of a microfilament component of a medical device, an electrospun micro-/nano-fiber-based construct, coated, twisted multifilament yarn, coated braided multifilament sutures, monofilament sutures, a molded internal bone-fixation device, a knitted mesh, a woven mesh, a knitted tape, and a woven tape.

11. The color-fast, sterilized surgical implant set forth in claim 1 wherein the radiation source comprises gamma rays.

12. The color-fast, sterilized surgical implant set forth in claim 1 wherein the radiation source comprises an electron beam.

13. The color-fast, sterilized surgical implant set forth in claim 1 wherein the radiation source is derived from a cold gas plasma.

14. The color-fast, sterilized surgical implant set forth in claim 1 wherein the step of sterilizing the implant is achieved radiochemically by a combination of high energy radiation and radiolytically generated formadehyde.

15. The color-fast, sterilized surgical implant set forth in claim 14 wherein the high energy radiation comprises gamma rays.

16. The color-fast, sterilized surgical implant set forth in claim 14 wherein the high energy radiation comprises an electron beam.

17. The color-fast, sterilized surgical implant set forth in claim lwherein the lipophilic dye comprises D&C Yellow 11 as described in the Color Index under C.I. number 47,000.

18. A color-fast, surgical implant comprising a polymer and a lipophilic, dispersed organic dye.

19. The color-fast, surgical implant set forth in claim 18 wherein the polymer comprises an absorbable/biodegradable polymer.

20. The color-fast, surgical implant set forth in claim 19 wherein the absorbable/biodegradable polymer comprises a polyester made from at least one cyclic monomer selected from the group represented by l-lactide, glycolide, p-dioxanone, 1,5-dioxepan-2-one, a morpholine-2,5-dione and ε-caprolactone.

21. The color-fast, surgical implant set forth in claim 20 wherein the polyester is made using a trifunctional initiator selected from the group consisting of trimethylolpropane, triethanolamine, and pentaerythritol.

22. The color-fast, surgical implant set forth in claim 19 wherein the absorbable/biodegradable polymer comprises a segmented/block copolymer.

23. The color-fast, surgical implant set forth in claim 22 wherein the segmented/block copolymer is made by grafting at least one cyclic polymer selected from the group consisting of l-lactide, glycolide, p-dioxanone, 1,5-dioxepan-2-one, a morpholine-2,5-dione and ε-caprolactone onto a polymeric initiator selected from the group consisting of poly(alkylene succinate) glycol, polyalkylene glycol, copolymeric polyalkylene glycol, and polytrimethylene carbonate glycol.

24. The color-fast, surgical implant set forth in claim 23 wherein the polymeric initiator is made by ring-opening polymerization of at least one monomer selected from the group consisting of trimethylene carbonate, ε-caprolactone, and glycolide in the presence of a trifunctional initiator selected from the group consisting of trimethylopropane, triethanolamine, and pentaerythritol.

25. The color-fast, surgical implant set forth in claim 18 wherein the polymer comprises a non-absorbable polymer.

26. The color-fast, surgical implant set forth in claim 25 wherein the non-absorbable polymer is selected from the group consisting of polypropylene, polyethylene terephthalate, segmented copolymers of polytetramethylene terephthalate and polytetramethylene oxide, segmented polycarbonate urethanes, and segmented polyether urethanes.

27. The color-fast, surgical implant set forth in claim 19 in the form of an absorbable thread comprising a medical device or component thereof selected from the group consisting of a microfilament component of a medical device, an electrospun micro-/nano-fiber-based construct, coated, twisted multifilament yarn, coated braided multifilament sutures, monofilament sutures, a molded internal bone-fixation device, a knitted mesh, a woven mesh, a knitted tape, and a woven tape.

28. The color-fast, sterilized surgical implant set forth in claim 18 wherein the lipophilic dye comprises D&C Yellow 11 as described in the Color Index under C.I. number 47,000.
